(19) United States
(12) Patent Application Publication
(10) Pub. No.: US 2004/0142492 A1
(43) Pub. Date: Jul. 22, 2004

(54) Method for detecting blood cell antigens and the antibodies in response to the same

(76) Inventors: Holger Kiesewetter, Berlin (DE); Abdulgabar Salama, Berlin (DE)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/477,151

(22) PCT Filed: May 10, 2002
(86) PCT No.: PCT/EP02/05140

(30) Foreign Application Priority Data
May 10, 2001 (DE) 101 22 806.6

(51) Int. Cl. G01N 33/543

(52) U.S. Cl. 436/518

(57) ABSTRACT

The invention relates to a method for detecting blood cell antigens and the antibodies in response to the same in a sample. The invention also relates to kits which facilitate the completion of said method.

The present invention relates to a method for detecting antigens on blood cells or for detecting antibodies directed against these antigens in human blood. This method is carried out in vitro using blood which is taken from the patient.

In the present-day diagnosis of particular diseases, it is necessary to detect selected antigens derived from red and white blood cells (erythrocytes and leukocytes, respectively) and from blood platelets, or else specific antibodies directed against these antigens, in the blood of the patients.

Blood cell antigens are surface markers (characteristics) on red blood cells (erythrocytes), blood platelets (thrombocytes) and white blood cells (leukocytes). They are usually glycoproteins, glycolipids or proteins. A summary in this regard can be found, for example, in Transfusionsmedizin [Transfusion medicine], Mueller-Eckhardt (Ed.), Springer-Verlag Berlin (1999), pp. 137-200.

The clinical importance of these blood cell antigens ensues from their immunogenicity, i.e. their ability to stimulate the formation of specific antibodies, for example after a transfusion, in an individual who does not possess these antigens under normal circumstances.

The formation of these antibodies directed against blood cell antigens can also be induced, for example, postnatally, as is the case with the blood group isoagglutinins (anti-A and anti-B), or by allogenic cells being transferred within the context of a pregnancy, an organ transplantation or a bone marrow transplantation. Examples of corresponding antibodies are anti-D, anti-K, anti-Fy, etc., directed against erythrocytes, anti-HPA-1 and anti-HPA-2, directed against platelets, anti-INA-1 and anti-INA-2, directed against granulocytes, or antibodies directed against HLA Class I and Class II.

In addition to this, antibodies can also be formed against endogenous antigens (autoantibodies). These antibodies can lead to the premature breakdown or destruction of autologous cells, as in the case of autoimmune hemolytic anemia, autoimmune thrombocytopenia or autoimmune neutropenia (agranulocytosis).

In fetuses and newborn infants, alloantibodies directed against erythrocytes, in particular immune antibodies such as anti-D and anti-c, can give rise to hemolysis (morbus haemolyticus neonatorum) of differing severity.

Isoagglutinins and clinically relevant alloantibodies have to be taken into account prior to any blood transfusion using erythrocytes. As a rule, no serologically untolerated erythrocytes, for example rhesus-positive erythrocyte concentrates when the (rhesus-negative) recipient possesses an alloantibody of the anti-D specificity, may be transfused. For this reason, it is necessary, prior to transfusions, to identify antibodies directed against erythrocytes and to take them into account during a transfusion. In addition, it is necessary, prior to any transfusion, to carry out a serological tolerance test (crosstest) using all the stored blood samples which are earmarked for the transfusion.

To date, antibodies directed against erythrocytic antigens have been detected routinely by incubating serum samples against native, selected test erythrocytes (antibody search and, where appropriate, antibody identification) or, prior to a transfusion, against donor erythrocytes (crosstest). The intolerance between serum and erythrocytes is manifested by direct or indirect agglutination of the erythrocytes under investigation. The isoagglutinins anti-A and anti-B, for example, induce direct agglutination of the erythrocytes. Indirect agglutination is induced, for example, by incubating anti-D with rhesus-positive test erythrocytes and then adding anti-human globulin serum (indirect Coombs test).

The disadvantages of using test erythrocytes arise from the selection, rarity and availability of given test erythrocytes and the short time during which the cells can be stored. In order to isolate the cells, it is regularly necessary to search for selected donors possessing known antigens, and the isolated cells can only be stored for a short period.

Virtually all the previously described test methods are based on using such cells (see Transfusionsmedizin [Transfusion medicine], Mueller-Eckhardt (Ed.), Springer-Verlag Berlin (1999), pp. 587-596 and New diagnostic methods in oncology and hematology, Huhn (Ed.) Springer Verlag, Berlin, 1998, pp. 197-212).

Disadvantages associated with these methods are therefore:

In order to recognize specific antibodies, it is necessary to use several (as a rule more than 11) test erythrocytes (test cells) per test since it is not possible to obtain erythrocytes which possess isolated (single) characteristics (e.g. cells which only carry the characteristic “D”).

As a rule, test erythrocytes which possess rare characteristics for recognizing particular antibodies are not available. It is not always possible to ensure that affected patients will be attended to.

As a rule, test erythrocytes which lack characteristics which normally occur frequently are not available for recognizing specific antibodies. For this reason, too, it is not always possible to ensure that affected patients are attended to.

It is an elaborate and expensive matter to select the appropriate test cells.

Since the material is biological material isolated from humans, the possibility of clinical personnel, doctors, etc., who are dealing with the material, being at risk of an infection is not always ruled out.

The cells can only be stored for a limited period.

The clinical importance of antibodies directed against thrombocytic antigens is reflected in autoimmune thrombocytopenia (AITP), neonatal alloimmunethrombo-cytopenia (NAIT), posttransfusional purpura (PTP) and the transfusion of incompatible platelets. In every case, the antibodies can give rise to a primary disturbance in hemostasis and hemorrhagic diathesis in the affected patients.

It has thus far been difficult to detect antibodies directed against platelets, and such detection can only be carried out in some specialist laboratories. Because of the nature of blood platelets, it has not so far been possible to develop a simple and rapid agglutination test, as in the case of erythrocytes. The previously described methods which are most frequently used are the enzyme-linked immunosorbent assay (ELISA) and the platelet adhesion immunofluorescence test (PIFT) and, for specifying the bound immunoglobulins, the immunoprecipitation or MAIPA (monoclonal antibody immunobilization of platelet antigens) assay (see Transfusionsmedizin [Transfusion medicine], Mueller-Eckhardt (Ed.), Springer-Verlag Berlin (1999) pp. 597-602, New diagnostic methods in oncology and hematology, Huhn (Ed.) Springer Verlag, Berlin (1998), pp. 213-228, McMillan, Transfusion Medicine Reviews, Vol. IV. No. 2, pp 136-143 (1990).

These methods suffer from a wide variety of disadvantages:

All the test methods are labor-intensive since standardized platelets are not as a rule available and the tests can only be carried out by experts.

It is frequently not possible to isolate and investigate, for example in an ELISA or MAIPA, any autologous platelets, or a sufficient number of autologous platelets, in the case of patients suffering from low platelet counts.

The specificity (ELISA and PIFT) and the sensitivity (MAIPA) are limited and, as a result, it is frequently not possible to interpret the results.

Essentially all the disadvantages which were described above in connection with detecting erythrocytic antigens also apply.

The clinical importance of antibodies directed against neutophil-dependent antigens is characterized by autoimmune neutropenia, neonatal alloimmune neutropenia and TRALI (transfusion-associated acute lung insufficiency). Detecting these antibodies is even more difficult than detecting thrombocytic antibodies. The previously known test methods are to a large extent adapted from methods for detecting antibodies directed against platelets (see Transfusionsmedizin [Transfusion medicine], Mueller-Eckhardt (Ed.), Springer-Verlag Berlin (1999), pp. 603-609, New diagnostic methods in oncology and hematology, Huhn (Ed.) Springer Verlag, Berlin, (1998), pp. 228-235, Minchinton and Waters, British Journal of Haematology, 56, pp. 521-528, (1984), McCullough and William, Transfusion Medicine Reviews, Vol. 1, No. 3, pp. 150-160 (1987)).

Furthermore, antibodies directed against lymphocytes, especially T lymphocytes, are of the greatest importance in transplantation immunology. These antibodies can bring about acute or delayed rejection of transplants (bone marrow, heart, lung, kidney and other organs). For this reason, it is necessary, before performing bone marrow and kidney transplantations, to rule out the presence of any lymphocytic antibodies directed against donor organs.

Furthermore, lymphocyte antigens (HLA antigens) can lead to the formation of specific antibodies directed against HLA characteristics in connection with pregnancies and transfusions. For this reason, the possible presence of these antibodies has to be taken into account in connection with transplantations and transfusions of platelets and leukocytes, where appropriate.

The classical method for detecting HLA antibodies has thus far been restricted to the lymphocytotoxic test. However, this test can only be used to determine complement-activating antibodies. The ELISA technique which has recently been used is extremely difficult and only implemented in some specialist laboratories (see Transfusionsmedizin [Transfusion medicine], Mueller-Eckhardt (Ed.), Springer-Verlag Berlin (1999), pp. 611-617, Zachary et al. Transplantation, Vol. 60, No. 12, pp 1600-1606 (1995), Lubenko and Rodi, Transfusion, Vol. 38, pp. 41-44 (1998)).

In summary, all the test principles for detecting antibodies directed against blood cells are based on using selected biological, and usually native, test cells which are obtained from selected individuals and then made available.

A relatively rapid and simple test is only possible for detecting customary antibodies directed against erythrocytes, for example detecting an anti-D antibody.

The search for alternatives has thus far been unsuccessful.

Although the genes for most blood group systems (erythrocytes, platelets and leukocytes) have by now been cloned, with some of them having been sequenced, recombinant antigens and peptides are only used in isolated cases, if at all, for detecting antibodies (Bowditch et al., Blood, Vol. 88, No. 12, pp. 4579-4584 (1996), Peterson et al., Blood, Vol. 92, No. 6, pp. 2053-2063 (1998), Yazdanbakhsh, Transfusion Medicine Reviews, Vol. 15, No. 1, pp. 53-66, (2001)).

Even the abovementioned MAIPA test, in which antibodies are immobilized on microtiter plates for the purpose of implementing an indirect ELISA test, still presents enormous practical problems in handling. Thus, this test lasts approx. 7-8 hours and the test results are frequently ambiguous. In addition, it is not possible to store the coated microtiter plates sufficiently long, with the result that all the appropriate solutions have in practice to be prepared freshly in the laboratory.

Meyer et al., THE LANCET Vol. 354, No. 9189, pp. 1525-1526 (1999) have described a test system which was used successfully to detect antibodies directed against the complex consisting of platelet factor 4 and heparin by adding patients' serum samples to antigen-coated beads in a particle agglutination test. Agglutinated samples were detected by means of a gel card test (see, for example, Salama and Mueller-Eckhard in: Transfusions-medizin [Transfusion medicine], Mueller-Eckhardt (Ed.), Springer-Verlag Berlin (1999), pages 587-617). However, despite their most strenuous efforts, the inventors have not succeeded in using this relatively rapid and easily implementable test to detect other blood cell antigens or antibodies directed against these antigens.

Despite the extremely great need, following inevitably from what has been said above, for a test system which can be used for detecting antibodies directed against selected blood cell antigens and which overcomes the above-listed disadvantages, no such test system has been made available prior to the present invention.

U.S. Pat. No. 6,203,706 discloses methods which are to be used for detecting blood cell antigens or antibodies in blood samples, for example. However, it has been found that the corresponding methods, in which antigens are bound directly to beads, only function in rare cases.

In view of the abovementioned prior art, the present invention was therefore based on the object of making available a method which can be used to specifically detect antibodies which are directed against selected blood cell antigens or to detect blood cell antigens themselves. This method should be simple and rapid in its operation and reliable and economical in its implementation. Furthermore, it should be possible to carry out the method in vitro on samples which can be obtained from living organisms.

This object, and other objects which are not explicitly mentioned but which can be readily deduced or inferred from the correlations which are discussed herein by way of introduction, are achieved by a method having all the features of patent claims 1-4. Expedient modifications of the novel method are protected in the appendant subclaims.

Variant 1:

By means, in an in-vitro method, of

(i) coating a bead with a nonhuman antibody which is directed against the specific blood cell antigen to be detected,

(ii) mixing appurtenant, specific, selected blood cell antigens, which can be obtained, where appropriate, by solubilizing standardized blood cells, which are already known per se in the prior art, or else by means of recombinant methods or methods of protein chemistry, with the coated beads obtained from (i),

(iii) mixing the beads, which are coated with antigen as a result of the binding of the selected antigen to the nonhuman antibody which is bound to the bead in (i), with a serum sample from a patient to be investigated, and

(iv) using a specific anti-human antibody test to detect the patient's blood cell antigen antibody which is derived from the sample and which may have bound to the antigen which is bound to the bead,

success is achieved, in a manner which is not readily foreseeable, in making available a method for detecting, in a sample, antibodies directed against blood cell antigens, with the method enabling the detection to be effected readily. At the same time, this method exhibits the abovementioned advantages as compared with the prior art.

Variant 2:

It only denotes a slight modification of the novel method for detecting antibodies directed against blood cell antigens when

(i) beads are coated with non-anti-human, species-specific antibodies,

(ii) standard cells are solubilized, or blood cell antigens which have been obtained recombinantly or using methods of protein chemistry are brought into solution,

(iii) the solubilizate, or the solution of blood cell antigens which have been obtained recombinantly or using methods of protein chemistry, resulting from (ii) is incubated with specific antibodies which are directed against the antigen to be detected and are derived from the species defined in (i),

(iv) the complexes, which have been formed in (iii) with the beads from (i), are isolated by centrifugation, and

(v) investigated directly or subsequently, in an agglutination test, against antibody-containing serum derived from patients.

Variant 3:

Success is also achieved by, in an in-vitro method,

(i) coating a bead with an antibody which is directed against the specific blood cell antigen to be detected,

(ii) subjecting blood samples derived from the patient to be investigated, together with the blood cells contained therein, to a treatment by which the antigens present in the membrane of the blood cells are solubilized, and preparing an antigen-rich fraction, where appropriate,

(iii) mixing the coated beads obtained from (i) with the sample obtained from (ii),

(iv) using a specific antibody test to detect the blood cell antigen which is derived from the sample and which may have bound to the antibody which is bound to the bead.

Variant 4:

It is furthermore possible to detect blood antigens in samples by

(i) coating beads with non-anti-human, species-specific antibodies,

(ii) solubilizing blood cells which are derived from patients to be investigated,

(iii) incubating the solubilizate resulting from (ii) with specific antibodies which are directed against the antigen to be detected and are derived from the species defined in (i),

(iv) isolating the complexes, which have been formed in (iii) with the beads from (i), by centrifugation, and

(v) investigating the sample in an agglutination test.

In particular, the novel methods are:

simple and rapid in their operation, and

reliable and economical in their implementation.

Whereas it was previously almost always necessary to use native test cells, or to prepare native test cells at a point close in time, in order to implement the systems which have thus far been described in the prior art, the novel method can dispense with this. In particular, the ready-coated beads which can be used in accordance with the invention provide substantially higher storability than native test cells. In addition, these coated beads can in theory be provided in unlimited quantity and kept for the time when the need arises, resulting in a substantial simplification for this reason as well.

It is essential for the present invention that blood cell antigens, or antibodies which are directed against them, are detected by the native antigens being fished out of a sample using the monoclonal antibodies which are directed against them. It is only when using this method modification, which is essential when compared with the prior art, that the abovementioned technical object is successfully achieved so readily and reliably and with little input.

Ready-coated beads which can be used in accordance with the invention are the beads obtained from step (ii) in variant 1, the beads obtained from step (iv) in variant 2, the beads obtained from step (i) in variant 3 and the beads obtained from step (i) in variant 4.

These beads can be stored for a long time, i.e. substantially longer than in the case of native test cells.

Since the biological starting material can be prepared in specialist laboratories which are specifically geared for this purpose, the risk of the personnel involved becoming infected is also reduced.

The invention involves detecting blood cell antigens or antibodies which are directed against them. At present, more than 700 different erythrocytic antigens have been described. Very many more antigens have been reported to be present in other blood cells. All these antigens possess, or may possess, clinical relevance. It is therefore clear to the skilled person that the present invention can in principle be used to detect all these antigens rapidly and reliably.

A compilation of these blood cell antigens can be found, for example, in Issitt, P. and Anstee, D. (Ed.): APPLIED BLOOD GROUP SEROLOGY, Montgomery Scientific Publications, Durham, N.C., USA. Particularly important blood group antigens which are preferably detected using the present invention are: AB0; C, C^{W}, c, D, E, e, K, k, Fy (a), Fy (b), Jk (a), Jk (b), S, s, M, N, P (1), Le (a), Le (b).

Very many of the antibodies which are also required are already available. Examples of these antibodies are: anti-Jka (from DiaClon), anti-Jkb (from DiaClon), anti-Lea (DiaClon), anti-Leb (DiaClon), Rh test sera from BIOLITH DIAGNOSTIKA, Hann. Münden, Germany (anti-D, anti-C, anti-c, anti-C^{w}, anti-E, anti-e, test sera of the MNSs system (anti-M, anti-N, anti-S) or else the sera supplied by Dade (e.g. anti-K₁). In all, more than 600 different monoclonal antibodies directed against blood cell antigens are commercially available world-wide.

In cases where such antibodies are not available, they can be prepared either polyclonally or monoclonally. The skilled person is very familiar with methods for preparing antibodies.

The protocols which are also used in the methods which are already described in the prior art can be employed for solubilizing the blood cells or their antigens. As has already been explained elsewhere, a very important advantage of the present invention is that, due to the substantially greater durability of the coated beads, these protocols can be carried out on a large scale in laboratories which are geared to them.

In order to prepare the coated beads which can be used in accordance with the invention, the specific antibodies have to be bound to the beads.

This binding is preferably mediated by way of a streptavidin or avidin/biotin complex, with biotinylated antibodies being bound to microcarriers which are coated with streptavidin or avidin. The corresponding methods are well known to the skilled person and described in detail in U.S. Pat. No. 6,203,706.

In particular, it is preferred when an antibody, which is biotinylated and which is directed against the antigen to be detected, initially reacts with the native cells (test cells), after which the cells are solubilized and the complex of antigen and biotinylated antibody which has been formed is removed from the reaction mixture using beads (microcarriers) which are coated with streptavidin or avidin.

This complex which has been formed can then be used in two different ways:

1.) If the presence of an antibody directed against the antigen is to be detected in a patient's serum, the complex which has been formed can then be incubated directly with the serum. Any antibodies which may be present then react with the antigen and bind to the complex. Since these antibodies to be detected are of human origin, the antibodies to be detected can be bound, after the complex has been separated off from the test mixture, using an anti-human antibody and employing well-known methods.

The following is a nonexhaustive list of possible methods: standard anti-human globulin test, card test (Y. Lapierre et al., (1990), Transfusion 30(2):109-113), ELISA (detection on a microtiter plate), microchip, capillary diffusion, flow cytometry, radio-immunoassay (RIA; e.g. iodine¹²⁵-labeling of the beads), etc. A whole series of methods are available to the skilled person. The gel card method (card test) and flow cytometry have proved to be particularly well suited.

2.) If the antigen is to be detected directly, the same methods can be used to directly bind a second antibody to the abovementioned complex composed of antigen and biotinylated antibody and streptavidin-coated or avidin-coated beads. For this, the second antibody is either directly labeled (e.g. iodine¹²⁵, enzymic labeling) or else it is detected once again by way of a species-specific antibody and using the above-mentioned methods, with the species-specific antibody having to be directed against species from which the second antibody is derived.

Particularly preferred variants of the novel system for detecting blood cell antigens, or antibodies directed against them, in serum samples therefore comprise:

An in-vitro method for detecting, in a patient's serum sample, antibodies directed against blood cell antigens, in which method

(i) biotinylated nonhuman antibodies which are directed against the antigen to be detected are brought into contact with human test cells which carry the corresponding, native blood cell antigens,

(ii) the complex formed from (i) is subjected to conditions under which the blood cells solubilize,

(iii) the complex composed of antigen and biotinylated antibody is removed from the sample using streptavidin-coated or avidin-coated microparticles,

(iv) the complex which has been formed in (iii) is brought into contact with a serum sample from a patient to be investigated, and

(v) an anti-human antibody test is used to detect the complexes which are formed from biotinylated antibody, antigen and human antibody from the serum when specific antibodies are present in the serum sample, once the complex has been separated off from the sample.

and also

an in-vitro method for detecting selected blood cell antigens, in which method

(i) biotinylated antibodies which are directed against the antigen to be detected are brought into contact with human test cells which carry the corresponding, native blood cell antigens,

(ii) the complex formed from (i) is subjected to conditions under which the blood cells solubilize,

(iii) the complex composed of antigen and biotinylated antibody is removed from the sample using streptavidin-coated or avidin-coated microparticles,

(iv) the complex which has been formed in (iii) is brought into contact with a preferably labeled second antibody which is directed against the antigen, and

(v) customary methods are used to detect the complexes which are formed from biotinylated antibody, antigen and second antibody once the complex has been separated off from the sample.

It is also possible for the antibody to be bonded chemically to the beads.

For the purposes of the present invention, beads are usually understood as being spherical microcarriers composed of different materials.

Preference is given, in this connection, to using ferromagnetic streptavidin particles (e.g. M280 streptavidin Dynabeads).

The physical properties of these particles are as follows:

Diameter: 2-4 mm

Density: 1.1-1.8 g/cm3

Surface area: 4-8 m2/g

Magnetic mass moment: 100±25×10−6 m3/kg

The suspension preferably contains 10 mg of particles/ml, corresponding to 6.7×10⁸/ml

The binding properties of the particles are preferably as follows:

1 mg of M280 streptavidin Dynabeads can bind 5-10 g of a biotinylated antibody (100% saturation of the streptavidin which is covalently bonded to the polystyrene surface)

Advantages of these particles are:

As a result of their paramagnetic properties, these particles are suitable for isolating biotin-antibody-antigen complexes from a heterogeneous mixture, such as a cell lysate, by simply using a magnet (magnetic particle concentrator).

However, these particles suffer from the following disadvantages:

No color-fluorescent particles are present.

There is no possibility of analysis by flow cytometry.

Red-fluorescing high-density streptavidin polystyrene particles, which are well known and commercially available, are therefore particularly preferably used in accordance with the invention.

The physical properties of these particles are as follows:

Diameter: 2-4 mm

Density: 1.1-1.8 g/cm³

The binding properties of these particles are preferably as follows:

Streptavidin density and behavior with regard to biotin antibody binding are the same as for Dynabeads (see above).

Advantages of these particles are

They fluoresce red, thereby facilitating readout.

Quality can also be monitored by flow cytometry.

Taken overall, the particles should preferably be composed of polystyrene and have a diameter of from 0.01 to 10 μm, particularly preferably 2-4 μm. It is particularly preferred for these microparticles to carry epoxy, carboxyl and/or amino radicals, or tosyl groups, on their surface.

However, it is clear to the skilled person that the microparticles which can be used in accordance with the invention can also be composed of a large number of different materials. The skilled person is very familiar with the field of microparticle technology and there are many different commercially available micro-particles which can potentially be used in accordance with the invention. The preferred size of the microparticles is given above.

After the second antibody has bound to the bead-first antibody-antigen complex, this second antibody is detected. For this purpose, either this second antibody can be fluorescence-labeled or enzymically labeled, for example, or else it is possible to use a labeled third antibody which is directed against this second antibody. There is no need to label the antibody if an agglutination test is carried out.

Such tests and detection methods are well known to the skilled person. Agglutination tests and flow cytometry can preferably be used in accordance with the invention.

It is a characteristic of the present invention that the individual components of the novel detection method are known to the skilled person. However, it was in no way possible to foresee that a combination of these individual steps would lead to a method which provides, in such an extremely surprisingly advantageous manner, a simple solution to a need felt so intensively by the scientific community.

In particular, it is also possible, according to the invention, to provide kits for detecting blood cell antigens or antibodies directed against blood cell antigens, which kits at least comprise spherical beads having a diameter of from 0.01 to 10 μm, preferably 2-4 μm, a specific antibody directed against a blood cell antigen and a specific anti-human antibody.

In another preferred embodiment, the kit for detecting blood cell antigens or antibodies directed against blood cell antigens at least comprises spherical beads having a diameter of from 0.01 to 10 μm, preferably 2-4 μm, a first non-anti-human, species-specific antibody and also an antibody which is directed against the antigen to be detected and which is derived from the species against which the first antibody is directed.

It is clear that other kits which contain the essentially required constituents which are described in the methods according to the invention also form part of the subject matter of the application.

The following examples explain the invention in more detail. However, they should not be understood as being in any way limiting.

### EXAMPLE 1

### Solubilizing Cells

Solubilization buffer: 1.21 g of Tris

to 950 ml of NaCl

pH 7.4

addition of 5 ml of Triton X-100

to 1000 ml of NaCl

The test cells (platelets) are sedimented at 10 000 rpm for 1 min in a bench centrifuge (manufacturer: Hettich). The supernatant is aspirated off and the cell pellet is taken up in 100 μl of solubilization buffer.

### EXAMPLE 2

A specific anti-GPIIa-IIIb antibody (from BioTest) was biotinylated and treated in solution with streptavidin-coated beads. The antibody was shown to have bound to the beads. In this connection, it was found that the presence of 1 mM zinc promotes the binding.

Subsequently, the beads are incubated with solubilized blood cells and a fluorescence-labeled second antibody is added. A positive test assay is characterized by an increase in the attachment of the secondary antibody to the complex, which could in turn be measured by way of the fluorescence, which was imparted thereby, of the isolated bead-first antibody-antigen-second antibody complex.

In this way, it was possible to detect the GPIIa-IIIb antigen in the blood in an amazingly simple manner.

### EXAMPLE 3

An agglutination test (gel card test from Diamed, Switzerland) was carried out instead of a test based on fluorescence labeling.

In this way, too, it was possible to detect the GPIIa-IIIb antigen in the blood in an amazingly simple manner.

### EXAMPLE 4

Instead of using an Elisa test, the second antibody-labeled beads were detected by means of flow cytometry.

In this way, too, it was possible to detect the GIIa-IIIb antigen in an amazingly simple manner.

### EXAMPLE 5

Carrying out a test according to the invention using streptavidin particles supplied by Diamed.

Patients/Donors and Sample Material

20 μl of serum (inactivated at 56° C. for 30 min)

Reagents and Equipment

Red-Fluorescing High-Density Streptavidin Polystyrene Particles (Diamed)

Physical properties:

Diameter: 2-4 μm

Density: 1.1-1.8 g/cm³

The suspension contains 0.075% particles (v/v); for this reason, these beads were used in a volume of 50 μl (as compared with 10 μl in the case of Dynabeads)

Binding Properties:

Streptavidin density and behavior with regard to biotin antibody binding are the same as in the case of Dynabeads.

Advantages: the particles fluoresce red; this facilitates readout and quality can also be monitored using the method of flow cytometry, which is well known to the skilled person.

Disadvantages: it appears to be necessary to block the particles with PBS-BSA after coating since these particles continue to exhibit high absorptivity.

Buffers

| | |
|---|---|
| Particle buffer | 10 mM PBS solution, pH 7.4, 0.1% |
| | Tween 20 |
| PBS-BSA (2% strength): | Dulbecco's phosphate-buffered |
| | saline × 10 |
| | diluted 1:10 in distilled water |
| | pH 7.2 |
| | addition of 22% (1:10) bovine |
| | albumin |
| Solubilization buffer: | 1.21 g of Tris |
| | to 950 ml NaCl |
| | pH 7.4 |
| | addition of 5 ml of Triton X-100 |
| | to 1000 ml NaCl |

antibodies employed:

| | | | | | Biotin |
|---|---|---|---|---|---|
| | | | | | excess |
| Specificity | Clone | Species | Isotype | Form | factor |
| GP IIb/IIIa | P2 | Mouse | IgG1 κ | purified | 15 |
| (CD41) | | | | | |
| GP Ia/IIa | Gi9 | Mouse | IgG1 κ | purified | 15 |
| (CD49b) | | | | | |
| GP Ib/IX (CD | FMC-25 | Mouse | IgG1 κ | purified | 15 |
| 42a) | | | | | |
| GP IV (CD 36) | FA6-152 | Mouse | IgG | purified | 15 |
| CD 9 | ALB 6 | Mouse | IgG1 κ | purified | 15 |
| Mouse IgG Fcγ | | Goat | | purified | |
| frag. | | | | | |
| Human IgG Fcγ | | Goat | | peroxidase | |
| frag. | | | | | |

Biotin: Sulfo-NHS-LC-biotin, 25 mg, Pierce, Perbio Science, Rockford, USA

Filtration after biotinylation in Millipore Ultrafree-MC 30 filter units (Millipore Corporation, Bedford, USA), filtration limit 30 kDa

Membrane: low-binding regenerated cellulose

Calibration:

Spontaneous agglutination is tested and negative controls are included. Each new batch or coupling is checked quantitatively in a flow-cytometric analysis. In connection with this, antigen-coated particles are incubated with a fluorescence-labeled antibody which is specifically directed against the antigen. The fluorescence intensity on the surface of the particles, which intensity can be measured readily by flow cytometry, is proportional to the degree of coating.

Quality control: inclusion of positive and negative control sera

Procedure:

Preparation

Biotinylating the Monoclonal Antibodies

Biotin is a vitamin (244 daltons) which functions as a cofactor for the carboxylases. As a result of its small size, biotin does not, after having been bound, as a rule have any influence on the properties of a macromolecule. The reactive groups for biotin are the ε-amino groups of lysine and tyrosine (NHS-biotin), with the formation of an amine bond. Since antibodies can also contain lysine in the area of the variable regions, the biotinylation can lead to a loss of antigen detection. In order to exclude or reduce this effect, an optimal biotin concentration (biotin excess) at which the functional regions of the antibody binding are not disturbed (Ternyck and Avrameas 1990), has to be found for each antibody. The use of NHS-LC-biotin has been found to be advantageous for macromolecules such as glycoproteins. In this case, any possible steric hindrances of the macromolecules is prevented by an additional chain containing 6 carbon molecules (hexanoate spacer, 22 A). After the Sulfo-NHS-LC-biotin has been brought into solution, rapid processing is to be recommended since the dissolved biotin is very susceptible to hydrolysis.

Procedure:

Dissolve the protein in 1 ml of PBS and calculate mmols of the biotin excess (factor 10-15): mmoles of protein×(12 or 20)=mmoles of biotin reagent added: mmoles of biotin to be pipetted×556*=mg of biotin reagent to be added

(*556 is the MW of Sulfo-NHS-LC-biotin) calculation: mg of protein/MW of protein=mmoles of protein

e.g. 1.3/150 000=8.7×10⁻⁶ mmol of IgG

8.7×10⁻⁶×20=1.73×10⁻⁴ mmol of biotin reagent 1.73×10⁻⁴×556=0.096373 mg of biotin (0.1 mg)

1. Allow the Sulfo-NHS-LC-biotin to reach room temperature before opening.

2. Dissolve 1 mg of Sulfo-NHS-LC-biotin in 1000 μl of dH₂O (1 μg=1 μl).

3. Incubation for 30 min at room temperature (alternatively 2 hours of incubation in ice).

4. Remove the unconjugated biotin by centrifuging using a Millipore MC 30 filter in PBS.

5. The centrifuge setting varies in dependence on the protein; as a rule, 10-15 minutes at 10 000 rpm are sufficient; alternatively, 30 min at 3500 rpm

6. Store the biotinylated antibody at 4° C. in 0.01% sodium acid.

Calculating the Degree of Biotinylation:

The degree of biotinylation is determined photometrically using the HABA reagent (2-hydroxyazobenzene-4-carboxylic acid). For this, the absorption of the HABA reagent (360 μl) at 500 nm is firstly measured on its own and noted in order, then, to add the biotinylated sample (40 μl). 5 minutes after the addition, the absorption is determined once again at 500 nm. As a result of competitive binding in the HABA molecule, a color change takes place in dependence on the biotin content of the sample; i.e. the degree to which the sample is biotinylated is directly proportional to the ΔA.

1. A500=(0.9)×(A500 of the HABA reagent)−(A500 of the biotinylated protein)

2. mmol of the biotinylated protein/ml=mg of biotinylated protein/ml of the sample

3. μmoles of biotin/ml of reaction mix=A500

As an alternative to the HABA reagent test, it is possible to construct a biotinylation calibration curve for each protein employed. Using this curve, it is possible to determine, uniquely per protein and batch, the optimal biotin quantity, which is different for each protein.

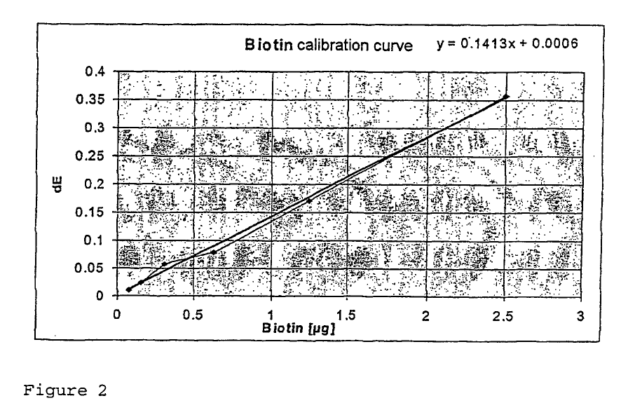

A calibration curve of this nature is shown in FIG. 2.

Sera: thaw control sera from −20° C. carefully, initially at 4° C.; centrifuge (2 min at 13 000 rpm; Eppendorf centrifuge)

Labeling and solubilizing the platelet glycoproteins Centrifuge 2×10⁷ test cells (standardized) at 10 000 rpm for 1 min in an Eppendorf tube, aspirate the supernatant, resuspend the cell pellet in 30 μl of PBS-2% BSA

10 μl of glycoprotein-specific biotinylated monoclonal antibodies are added per assay; an optimal dilution for each antibody employed must be determined beforehand by titrating for biotinylation (see Ruling out Spontaneous Agglutination) CD 41 1:60, CD 42a and CD49b 1:10, HLA antibody 1:200! Variations are batch-dependent!

Incubate at 37° C. for 30 minutes

Wash test platelets 3 times with 100 μl of PBS (centrifuge at 10 000 rpm for 1 min, aspirate off supernatant and take up platelet pellet in 100 μl of PBS.

After the last wash, lyse the pellets in in each case 100 μl of solubilization buffer; mix individually with a pipette

Incubate at 4° C. for 30 min

Isolation of antigen on streptavidin polymer particle

Centrifuge lysates at 13 000 rpm for 30 min (4° C., cell membrane residues are removed)

While the lysates are being centrifuged, wash the DiaMed streptavidin particles once with particle buffer at 10 000 rpm for 1 min

Add 10 μl of particles (concentration, 0.5%) to 70 μl of the platelet lysate

Incubate at 37° C. for 30 min

Wash the particles once with 100 μl of particle buffer, 10 000 rpm for 1 min,

Aspirate off supernatant

The particles are coated with antigen and can be introduced into the test system after having been resuspended in particle buffer to give a concentration of 0.075%.

Test Procedure for Antigen/Antibody Detection

Add 20 μl of serum to the particle pellet, resuspend the pellet well

Incubate at 37° C. for 30 min

Add 100 μl of particle buffer to the particles by pipette

Wash twice with in each case 100 μl of particle buffer

Take up in 50 μl of particle buffer, mix the pellet carefully

Pipette the particle suspension directly onto the goat anti-human card (Diamed; explained in Y. Lapierre et al., (1990), Transfusion 30(2):109-113)

Centrifuge the card, without any further incubation, in a DiaMed card centrifuge at 900 rpm for 10 min

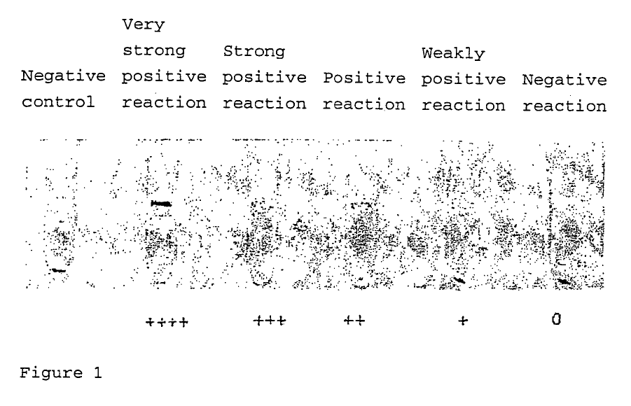

Assessment of results, see FIG. 1.

The high sensitivity of the test is clearly evident.

Thus far, 34 sera of differing reactivity have been tested selectively. Comparison with MAIPA shows that senstivity and specificity are comparable.

In N=8 sera, it was possible to detect the anti-Pla 1 specificity in parallel

In N=2 sera, it was not possible, despite a prior finding, to detect any anti-Pla 2 either in MAIPA or in the card test

In N=4 sera, it was possible to detect antibodies having anti-Bra specificity

In N=4 sera, it was possible to detect autoantibodies of varying specificity

N=8 sera were tested negative in MAIPA and the card test

N=8 sera reacted nonspecifically in both tests

With a test duration of 30-45 minutes (using previously coated particles), the labor and time input is minimal.

Presentation of Results:

Assessment (see FIG. 1)

Positive: agglutinated beads form a red line on the gel or are partitioned in the gel (++++ to +)

Negative: compact particle sediment at the bottom of the cavity in the ID card

1. An in-vitro method for detecting, in human samples, antibodies directed against blood cell antigens, characterized in that
(i) a bead is coated with a nonhuman antibody which is directed against the specific blood cell antigen to be detected,
(ii) appurtenant, specific, selected blood cell antigens, which can be obtained, where appropriate, by solubilizing standardized blood cells, which are already known per se in the prior art, or else by means of recombinant methods or methods of protein chemistry, are mixed with the coated beads obtained from (i),
(iii) the beads, which are coated with antigen as a result of the binding of the selected antigen to the nonhuman antibody which is bound to the bead in (i), are mixed with a serum sample from a patient to be investigated, and
(iv) a specific anti-human antibody test is used to detect the patient's blood cell antigen antibody which is derived from the sample and which may have bound to the antigen which is bound to the bead,

2. An in-vitro method for detecting antibodies directed against blood cell antigens, characterized in that
(i) beads are coated with non-anti-human, species-specific antibodies,
(ii) standard cells are solubilized, or blood cell antigens which have been obtained recombinantly or using methods of protein chemistry are brought into solution,
(iii) the solubilizate, or the solution of blood cell antigens which have been obtained recombinantly or using methods of protein chemistry, resulting from (ii) is incubated with specific antibodies which are directed against the antigen to be detected and are derived from the species defined in (i),
(iv) the complexes, which have been formed in (iii) with the beads from (i), are isolated by centrifugation, and
(v) investigated directly or subsequently, in an agglutination test, against antibody-containing serum derived from patients.

3. An in-vitro method for detecting blood cell antigens, characterized in that
(i) a bead is coated with an antibody which is directed against the specific blood cell antigen to be detected,
(ii) blood samples derived from a patient to be investigated, together with the blood cells contained therein, are subjected to a treatment by which the antigens present in the membrane of the blood cells are solubilized, and an antigen-rich fraction is prepared, where appropriate,
(iii) the coated beads obtained from (i) are mixed with the sample obtained from (ii),
(iv) a specific antibody test is used to detect the blood cell antigen which is derived from the sample and which may have bound to the antibody which is bound to the bead.

4. An in-vitro method for detecting blood cell antigens, characterized in that
(i) beads are coated with non-anti-human, species-specific antibodies,
(ii) blood cells which are derived from patients to be investigated are solubilized,
(iii) the solubilizate resulting from (ii) is incubated with specific antibodies which are directed against the antigen to be detected and are derived from the species defined in (i),
(iv) the complexes, which have been formed in (iii) with the beads from (i), are isolated by centrifugation, and
(v) the sample is investigated in an agglutination test.

5. An in-vitro method for detecting, in a patient's serum sample, antibodies directed against blood cell antigens, characterized in that
(i) biotinylated nonhuman antibodies which are directed against the antigen to be detected are brought into contact with human test cells which carry the corresponding, native blood cell antigens,
(ii) the complex formed from (i) is subjected to conditions under which the blood cells solubilize,
(iii) the complex composed of antigen and biotinylated antibody is removed from the sample using streptavidin-coated or avidin-coated microparticles,
(iv) the complex which has been formed in (iii) is brought into contact with a serum sample from a patient to be investigated, and
(v) an anti-human antibody test is used to detect the complexes which are formed from biotinylated antibody, antigen and human antibody from the serum when specific antibodies are present in the serum sample, once the complex has been separated off from the sample.

6. An in-vitro method for detecting selected blood cell antigens, characterized in that
(i) biotinylated antibodies which are directed against the antigen to be detected are brought into contact with human test cells which carry the corresponding, native blood cell antigens,
(ii) the complex formed from (i) is subjected to conditions under which the blood cells solubilize,
(iii) the complex composed of antigen and biotinylated antibody is removed from the sample using streptavidin-coated or avidin-coated microparticles,
(iv) the complex which has been formed in (iii) is brought into contact with a preferably labeled second antibody which is directed against the antigen, and
(v) customary methods are used to detect the complexes which are formed from biotinylated antibody, antigen and second antibody once the complex has been separated off from the sample.

7. A method as claimed in one of the preceding claims, in which fluorescence-labeled or enzymically labeled antibodies are used.

8. A method as claimed in one of the preceding claims, in which biotinylated antibodies are bound by way of streptavidin-coated and/or avidin-coated beads.

9. A method as claimed in one of the preceding claims, characterized in that the beads are spherical and have a diameter of from 0.01 to 10 μm, preferably from 2 to 4 μm.

10. A kit for detecting blood cell antigens or antibodies directed against blood cell antigens, comprising at least beads as claimed in claim 9, a specific antibody directed against a blood cell antigen, and also a specific anti-human antibody.

11. A kit for detecting blood cell antigens or antibodies directed against blood cell antigens, comprising at least beads as claimed in claim 9, a first non-anti-human, species-specific antibody and also an antibody which is directed against the antigen to be detected and which is derived from the species against which the first antibody is directed.
